(19) United States
(12) Patent Application Publication
(10) Pub. No.: US 2026/0166204 A1
(43) Pub. Date: Jun. 18, 2026

(54) Adaptive Responsive Materials for Medical Applications

(71) Applicant: Becton, Dickinson and Company, Franklin Lakes, NJ (US)

(72) Inventors: Mildred Calistri-Yeh, Killingworth, CT (US); Raghavendra Barki, Haveri (IN); Dirk Jan Broer, Geldrop (NL); Danqing Liu, Eindhoven (NL); Yuanyuan Zhan, Eindhoven (NL)

(21) Appl. No.: 19/126,672

(22) PCT Filed: Oct. 30, 2023
(86) PCT No.: PCT/US2023/078135

§ 371 Date: May 2, 2025

(30) Foreign Application Priority Data
Nov. 5, 2022 (IN) 202211063235

(51) Int. Cl. A61L 31/10

(52) U.S. Cl. CPC A61L 31/10

(57) ABSTRACT

Provided herein is a medical article including a substrate, a polymeric coating arranged on the substrate, the coating including a liquid crystal acrylate and a responsive component, and a biocompatible liquid received within the polymeric coating.

### CROSS-REFERENCE TO RELATED APPLICATION

The present application claims the benefit of Indian Provisional Patent Application No. 202211063235, filed Nov. 5, 2022, the content of which is incorporated herein by reference in its entirety.

### BACKGROUND OF THE INVENTION

### Field of the Invention

Disclosed herein are materials suitable for use in medical applications, and, in particular, porous polymeric materials that can absorb and release various substances in a controlled manner.

### Description of Related Art

Disinfection is of significant importance in the medical field, including both in intravenous access and in wound care. In terms of intravenous access, disinfection and ensuring the presence of catheter lock solutions are important because infections and clots in obstructed devices can rapidly become life-threatening. Today, separate manual steps are taken for cleaning connectors, disinfecting lumens, and introducing lock solutions. Each step requires diligence and care to be done well. It is challenging in the hospital environment and even more so in alternate care settings.

The presence of bacteremia originating from an I.V. catheter is characterized as a catheter-related bloodstream infection (CRBSI), sometimes known as catheter-related sepsis. Bacterial contamination of intravascular catheter infusion hubs, such as needle-free connectors (NFC) and locks, can result in CRBSI. Intravascular catheter entry ports are often contaminated with bacteria prior to the development of bloodstream infections. Contaminations of this type occur during the handling of intravascular line connectors during the connecting of infusions, drug injections, or blood sample withdrawal.

To reduce the introduction of intraluminal microbes via needle-free connectors, the Infusion Therapy Standards of Practice (INS) (8th edition, 2021) recommend either active disinfection with 70% isopropyl alcohol or alcohol-based chlorhexidine swab pads, or passive disinfection with disinfecting caps. Both active disinfection with alcohol-based chlorhexidine gluconate pads and passive disinfection with disinfecting caps were associated with lower rates of catheter-associated bloodstream infection (CABSI), while 70% isopropyl alcohol (IPA) swabs were found to be the least effective based on a meta-analysis of quasi-experimental studies. Furthermore, INS reported that alcohol-impregnated sponge devices are ineffective for decontamination a stopcock's internal lumen. In addition, INS recommended to replace the needle-free connector every 96-hour interval or according to the manufacturer's specification. According to INS, changing to a more frequent time interval added no benefit and may increase the risk of CABSI.

With regard to wound care, medical adhesive tapes are used in a wide variety of medical applications, for example, including as parts of over-the-counter bandages used for minor wounds and cuts, medical tapes for securing gauze or holding medical devices to the body, dressings for wounds, and securement devices for stabilizing venous catheters. Depending on an application's requirements, tapes may be required to be water-proof and/or to have sufficient strength and resistance to pull-off due to motion/torque. In general, however, the tape will need to be removed for access and then another piece is applied after inspection, medication addition, or other medical intervention.

Medical Adhesive-Related Skin Injuries (MARSIs) cover any type of skin damage related to the use of medical adhesive products like tapes, wound dressings, medication patches, and wound closure materials. These injuries occur whenever the attachment between the skin and the adhesive is stronger within the cells of the epidermis, resulting in mechanical trauma, dermatitis, or other damage. Currently, opportunities to reduce this problem and these injuries are limited for susceptible patients. Current tapes are not reusable, and even though a patient may need to have his/her wound or catheter secured for several days, access may be needed multiple times per day. This also drives the desirability of multiple-use adhesive.

Accordingly, there is a need in the art for new materials that can be used to provide disinfection and increased comfort for patients.

### SUMMARY OF THE INVENTION

Provided herein is a medical article including a substrate, a polymeric coating arranged on the substrate, the coating including a liquid crystal acrylate and a responsive component, and a biocompatible liquid received within the polymeric coating.

Also provided herein is a method of making a medical article with a polymeric coating arranged on a surface thereof, including steps of mixing a liquid crystal acrylate, a responsive component, a carboxylic acid, a porogen, and a photoinitiator in a first solvent to provide a liquid monomer mixture, applying the liquid monomer mixture to a substrate, removing the solvent to provide a substrate with a monomer mixture thereon, polymerizing the monomer mixture to provide a substrate with a polymeric coating thereon, immersing the substrate with the polymeric coating thereon in a second solvent to remove the porogen, thereby providing a substrate with a porous polymeric coating, immersing the substrate with the porous polymeric coating in an alkaline solution, and immersing the porous polymeric coating in a biocompatible liquid, thereby providing a substrate with a porous polymeric coating loaded with a biocompatible liquid.

Also provided herein is A method of using a medical article, the medical article including a substrate, a polymeric coating arranged on the substrate and having at least one liquid crystal acrylate and a responsive component, the polymeric coating including a plurality of pores, and a biocompatible liquid, the polymeric coating configured to exhibit a first orientation in which the biocompatible liquid is received within the pores, and a second orientation in which the biocompatible liquid is expelled from the pores, the method including exposing the medical article to ultraviolet light, thereby causing the polymeric material to exhibit the second orientation and expel the biocompatible liquid from the polymeric coating.

Additional non-limiting embodiments are set forth in the following numbered clauses:

Clause 1. A medical article comprising: a substrate; a polymeric coating arranged on the substrate, the coating comprising at least one liquid crystal acrylate and a responsive component; and a biocompatible liquid received within the polymeric coating.

Clause 2. The medical article of clause 1, wherein the polymeric coating is anisotropic.

Clause 3. The medical article of clause 1 or clause 2, wherein the liquid crystal acrylate comprises a liquid crystal diacrylate and a liquid crystal monoacrylate.

Clause 4. The medical article of any of clauses 1-3, wherein the liquid crystal diacrylate has the following structure:

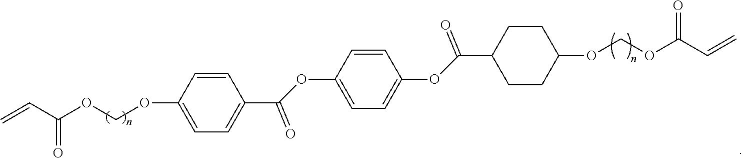

Clause 5. The medical article of any of clauses 1-4, wherein the liquid crystal diacrylate has the following structure:

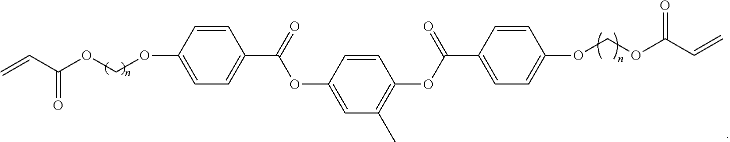

Clause 6. The medical article of any of clauses 1-5, wherein the liquid crystal monoacrylate has the following structure:

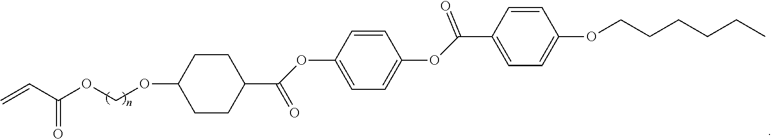

Clause 7. The medical article of any of clauses 1-6, wherein the liquid crystal monoacrylate has the following structure:

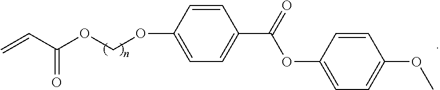

Clause 8. The medical article of any of clauses 1-7, wherein the responsive component is a light-responsive component comprising a light-responsive diacrylate.

Clause 9. The medical article of any of clauses 1-8, wherein the light-responsive component comprises an azobenzene moiety.

Clause 10. The medical article of any of clauses 1-9, wherein the light-responsive diacrylate has the following structure:

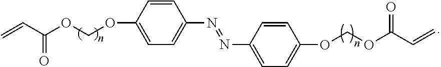

Clause 11. The medical article of any of clauses 1-10, wherein the polymeric coating further comprises a carboxylic acid.

Clause 12. The medical article of any of clauses 1-11, wherein the carboxylic acid is benzoic acid.

Clause 13. The medical article of any of clauses 1-12, wherein the polymeric coating further comprises a photoinitiator.

Clause 14. The medical article of any of clauses 1-13, wherein the photoinitiator contains phosphorous.

Clause 15. The medical article of any of clauses 1-14, wherein the photoinitiator has the following structure:

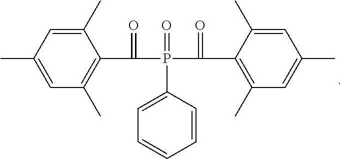

Clause 16. The medical article of any of clauses 1-15, wherein the polymeric coating comprises a plurality of pores configured to receive a liquid therein.

Clause 17. The medical article of any of clauses 1-16, wherein the biocompatible liquid is received within the pores of the polymeric coating.

Clause 18. The medical article of any of clauses 1-17, wherein the biocompatible liquid comprises one or more of polyethylene glycol, water, saline, dimethyl sulfoxide (DMSO), ethanol, isopropanol, and chlorhexidine gluconate.

Clause 19. The medical article of any of clauses 1-18, wherein the polymeric coating is configured to exhibit a first orientation in which the biocompatible liquid is received within the pores, and a second orientation in which the biocompatible liquid is expelled from the pores.

Clause 20. The medical article of any of clauses 1-19, wherein the polymeric coating is configured to exhibit the first orientation when exposed to visible light and to exhibit the second orientation when exposed to ultraviolet light.

Clause 21. The medical article of any of clauses 1-20, wherein the substrate comprises a catheter, a catheter hub, a luer connector, and/or a needle-free connector (NFC), and wherein the polymeric coating is arranged on an inner surface of the medical article.

Clause 22. The medical article of any of clauses 1-21, wherein the substrate is optionally flexible, and the medical article comprises an adhesive layer arranged on the polymeric coating, such that the polymeric coating is arranged between the adhesive and the flexible substrate.

Clause 23. The medical article of any of clauses 1-22, wherein the adhesive layer comprises a plurality of pores.

Clause 24. The medical article of any of clauses 1-23, wherein the adhesive layer comprises one or more of 2-ethylhexyl acrylate, acrylic acid, isobutyl methacrylate, poly (ethylene glycol) methyl ether methacrylate, and poly(ethylene glycol) diacrylate.

Clause 25. The medical article of any of clauses 1-24, wherein the substrate comprises one or more of a polyester, a polyurethane, an olefin, a polyamide, and acrylic polymer, an ethylene-vinyl acetate, a polyether, and derivatives thereof.

Clause 26. A method of making a medical article with a polymeric coating arranged on a surface thereof, comprising: mixing at least one liquid crystal acrylate, a responsive component, a carboxylic acid, a porogen, and a photoinitiator in a first solvent to provide a liquid monomer mixture; applying the liquid monomer mixture to a substrate; removing the solvent to provide a substrate with a monomer mixture thereon; polymerizing the monomer mixture to provide a substrate with a polymeric coating thereon; immersing the substrate with the polymeric coating thereon in a second solvent to remove the porogen, thereby providing a substrate with a porous polymeric coating; immersing the substrate with the porous polymeric coating in an alkaline solution; and immersing the porous polymeric coating in a biocompatible liquid, thereby providing a substrate with a porous polymeric coating loaded with a biocompatible liquid.

Clause 27. The method of clause 26, wherein the liquid monomer mixture is applied to the substrate by a cell method or a spin coating method.

Clause 28. The method of clause 26 or clause 27, wherein the first solvent is tetrahydrofuran (THF).

Clause 29. The method of any of clauses 26-28, wherein the second solvent is cyclohexane and/or ethanol.

Clause 30. The method of any of clauses 26-29, wherein the alkaline solution comprises potassium hydroxide (KOH).

Clause 31. The method of any of clauses 26-30, wherein the liquid crystal acrylate comprises a liquid crystal diacrylate and a liquid crystal monoacrylate.

Clause 32. The method of any of clauses 26-31, wherein the liquid crystal diacrylate has the following structure:

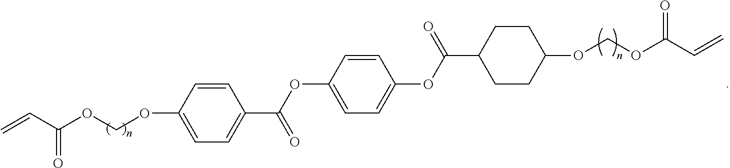

Clause 33. The method of any of clauses 26-32, wherein the liquid crystal diacrylate has the following structure:

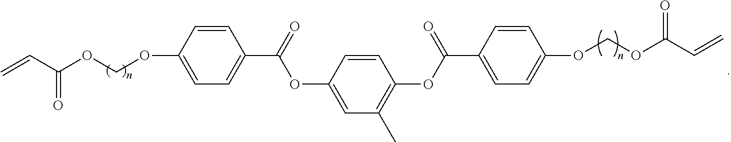

Clause 34. The method of any of clauses 26-33, wherein the liquid crystal monoacrylate has the following structure:

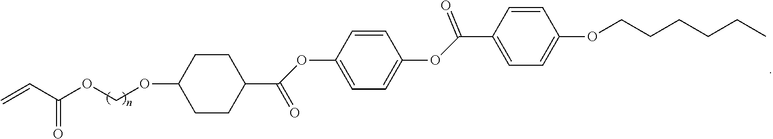

Clause 35. The method of any of clauses 26-34, wherein the liquid crystal monoacrylate has the following structure:

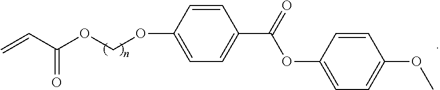

Clause 36. The method of any of clauses 26-35, wherein the responsive component is a light-responsive diacrylate.

Clause 37. The method of any of clauses 26-36, wherein the light-responsive component comprises an azobenzene moiety.

Clause 38. The method of any of clauses 26-37, wherein the light-responsive diacrylate has the following structure:

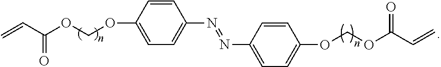

Clause 39. The method of any of clauses 26-38, wherein the carboxylic acid is benzoic acid.

Clause 40. The method of any of clauses 26-39, wherein the photoinitiator contains phosphorous.

Clause 41. The method of any of clauses 26-40, wherein the photoinitiator has the following structure:

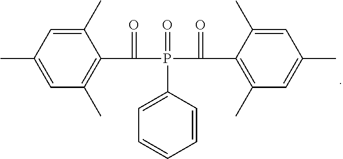

Clause 42. The method of any of clauses 26-41, wherein the biocompatible liquid comprises one or more of polyethylene glycol, water, saline, dimethyl sulfoxide (DMSO), ethanol, isopropanol, and chlorhexidine gluconate.

Clause 43. A method of using the medical article of any of clauses 1-25, the method comprising exposing the medical article to ultraviolet light, thereby causing the polymeric material to exhibit the second orientation and expel the biocompatible liquid from the polymeric coating.

### BRIEF DESCRIPTION OF THE DRAWINGS

FIG. 1 shows exemplary applications of a material according to non-limiting embodiments disclosed herein;

FIGS. 2A-2B show schematics of exemplary applications of a material according to non-limiting embodiments disclosed herein;

FIGS. 3A-3B are chemical structures for diacrylates useful in materials according to non-limiting embodiments disclosed herein;

FIGS. 4A-4B are chemical structures for methacrylates useful in materials according to non-limiting embodiments disclosed herein;

FIG. 5 is the chemical structure for a light-responsive diacrylate useful in materials according to non-limiting embodiments disclosed herein;

FIG. 6 is the chemical structure for a carboxylic acid useful in materials according to non-limiting embodiments disclosed herein;

FIGS. 7A-7B are chemical structures for porogens useful in materials according to non-limiting embodiments disclosed herein;

FIG. 8 is the chemical structure for a photoinitiator useful in materials according to non-limiting embodiments disclosed herein;

FIG. 9 shows chemical structures of the composition liquid crystal mixture of a material according to non-limiting embodiments disclosed herein;

FIG. 10 shows chemical structures of the composition liquid crystal mixture of a material according to non-limiting embodiments disclosed herein; and

FIG. 11 shows chemical structures of the composition liquid crystal mixture of a material according to non-limiting embodiments disclosed herein.

### DESCRIPTION OF THE INVENTION

For purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about”. Accordingly, unless indicated to the contrary, the numerical parameters set forth in the following specification and attached claims are approximations that may vary depending upon the desired properties to be obtained by the present invention.

Notwithstanding that the numerical ranges and parameters setting forth the broad scope of the invention are approximations, the numerical values set forth in the specific examples are reported as precisely as possible. Any numerical value, however, inherently contains certain errors, necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Provided herein are polymeric materials for use with medical articles, the materials allowing for secretion and uptake of biocompatible liquids that can be used in various medical applications. An advantage of the present polymeric materials, which exhibit switching (e.g., changing state or orientation, as will be described below), is that they are suitable for use with biocompatible liquids. Without wishing to be bound by the theory, it is believed that the polymeric materials described herein exhibit suitable polarity for use with such biocompatible liquids.

As used herein, the term “polymer” refers to oligomers and homopolymers (e.g., prepared from a single monomer species), copolymers (e.g., prepared from at least two different monomer species), terpolymers (e.g., prepared from at least three different monomer species) and graft polymers. The term “polymer” as used herein encompasses liquid crystal polymers. Herein below, the terms “polymeric material” and “liquid crystal polymer network (LCN)” are used interchangeably.

In non-limiting embodiments, the polymeric material as described herein includes one or more acrylates, such as alkyl or hydroxyalkyl esters of (meth)acrylic acid. Non-limiting examples of suitable acrylates include alkyl esters of (meth)acrylic acid, such as methyl (meth)acrylate, ethyl (meth)acrylate, butyl (meth)acrylate, isobutyl (meth)acrylate, ethylhexyl (meth)acrylate, lauryl (meth)acrylate, octyl (meth)acrylate, glycidyl (meth)acrylate, isononyl (meth)acrylate, isodecyl (meth)acrylate, vinyl (meth)acrylate, acetoacetoxyethyl (meth)acrylate, acetoacetoxypropyl (meth)acrylate, and combinations thereof. Non-limiting examples of hydroxyalkyl esters of (meth)acrylic acid include hydroxymethyl (meth)acrylate, hydroxyethyl (meth)acrylate, hydroxypropyl (meth)acrylate, hydroxybutyl (meth)acrylate, and combinations thereof. Herein below, “acrylate” and “liquid crystal acrylate” are used interchangeably. In non-limiting embodiments, useful acrylates, such as diacrylates, may cross-link in the formation of a polymer composition. In non-limiting embodiments, one or more acrylates as included in the polymeric materials described herein are attached to a mesogenic moiety, optionally to align the polymeric material.

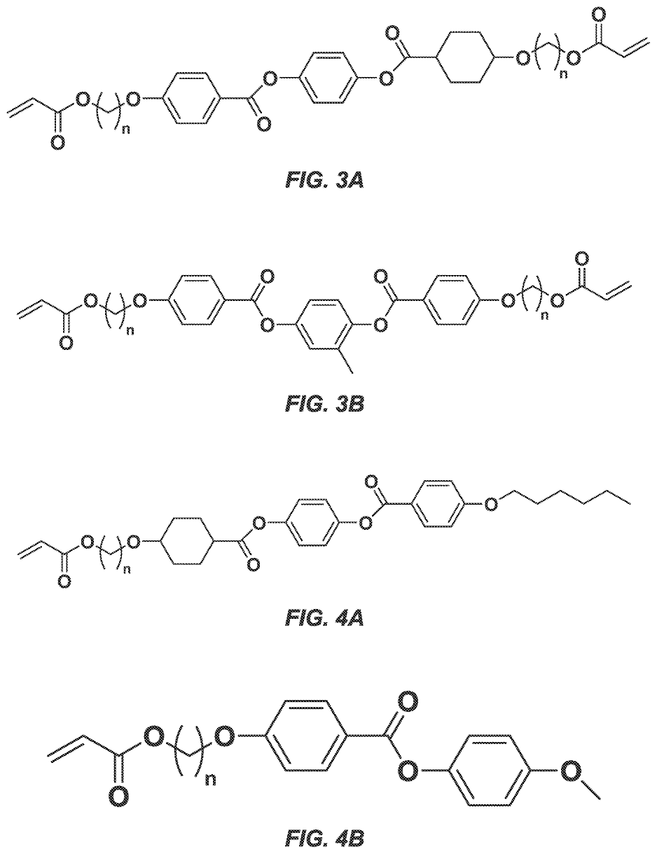

In non-limiting embodiments, the polymeric material includes one or more diacrylates and one or more monoacrylates. Non-limiting embodiments of suitable diacrylates and monoacrylates are shown in FIGS. 3A-4B, where n=1-99, optionally 3-11, all values and subranges therebetween inclusive.

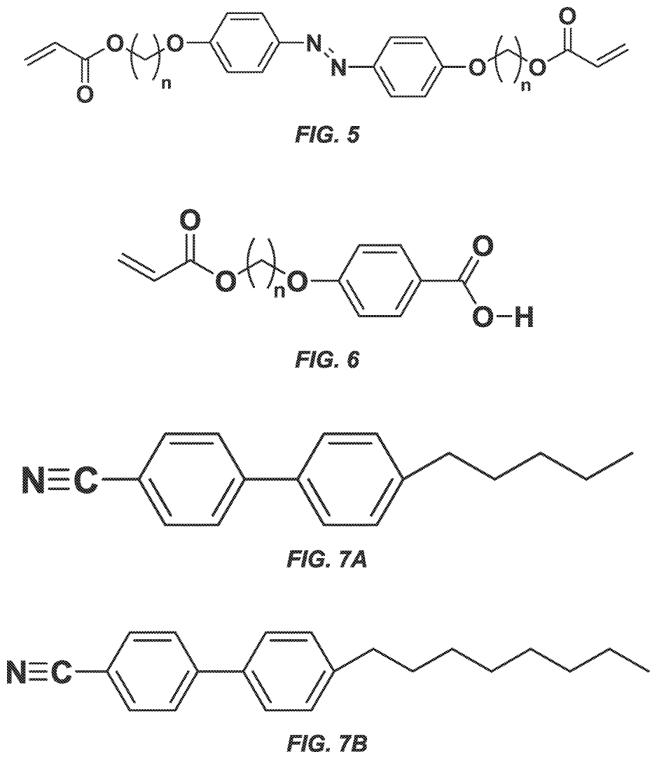

In non-limiting embodiments, the polymeric material as described herein includes a responsive component. As used here, “responsive component” refers to a component that can assume more than one orientation, depending on the type of stimulus (light, temperature, electrical stimulation) to which it is exposed. In non-limiting embodiments, the responsive component is a light-responsive component, in which the component switches orientation based on the type light to which it is exposed. For example, a component may assume a first orientation when exposed to visible light, and may assume a second orientation when exposed to ultraviolet (UV) light. In non-limiting embodiments, the responsive component includes an azobenzene moiety or a derivative thereof. In non-limiting embodiments, the light responsive component is a diacrylate. A non-limiting embodiment of a suitable diacrylate is shown in FIG. 5, where n=1-99, all values and subranges therebetween inclusive. In non-limiting embodiments, the responsive component is responsive to electrical stimulation, in which the component switches orientation based on the type/amount of electrical stimulation (e.g., frequency, voltage, and/or current) to which it is exposed. In a non-limiting embodiment, the responsive component is temperature responsive, switching orientation based on the amount of heat/temperature stimulation (e.g., elevated temperature, room temperature, and/or below room temperature) to which it is exposed.

In non-limiting embodiments, the polymeric material as described herein includes an acid, such as a carboxylic acid and its derivatives. Suitable carboxylic acids, include, but are not limited to, aromatic carboxylic acids, dicarboxylic acids, tricarboxylic acids, and combinations thereof. In non-limiting embodiments, the carboxylic acid is benzoic acid or a derivative thereof. A non-limiting embodiment of a suitable carboxylic acid is shown in FIG. 6, where n=1-99, all values and subranges therebetween inclusive.

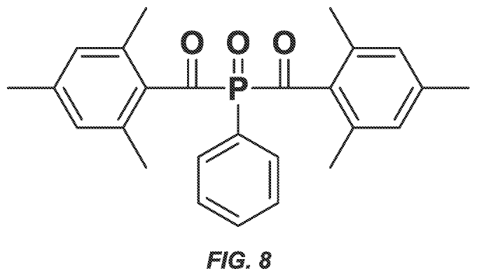

In non-limiting embodiments, the polymeric material is formed from a monomer mixture, such as a liquid monomer mixture, as will be described below. In order to aid in polymerization, in non-limiting embodiments the liquid monomer mixture includes a photoinitiator. In non-limiting embodiments, at least a residual amount of photoinitiator remains in the finalized polymeric material. Suitable photoinitiators (for example, those that are activated by light of wavelength in the range of 300 nm to 440 nm) are known to those of skill in the art, and may include Irgacure 819, commercially available from BASF (Ludwigshafen, Germany). A non-limiting embodiment of a suitable photoinitiator is shown in FIG. 8.

In non-limiting embodiments, the polymeric material as described herein is a porous material. In non-limiting embodiments, the pores of the polymeric material allow for a biocompatible liquid to be received and held within the polymeric material, and exposure to one type of light or another (as described briefly above). Polymeric materials with pores may be prepared by adding a porogen to the various monomer/polymer components described above, and later removing the porogen, for example by exposure to a solvent. Inorganic salts, sugars, polyvinyl acetate (PVA), polyethylene glycol (PEG), saccharose crystals, gelatin spheres, paraffin spheres, or any other non-reactive liquid-crystal molecules that phase separates during polymerization of the liquid crystal mixture are examples of suitable porogens. Non-limiting embodiments of suitable porogens are shown in FIGS. 7A-7B.

As described above, the polymeric materials described herein may be useful as vehicles for storage/delivery of biocompatible liquids, such as therapeutic compositions, for example by being received and/or held within pores of the polymeric material. Those of skill will appreciate that, depending on the application, any suitable biocompatible liquid may be included. In non-limiting embodiments, the biocompatible liquid includes an antiseptic, antibacterial, bactericidal, antifungal, and/or antiviral composition. In non-limiting embodiments, the biocompatible liquid includes one or more medications, such as, without limitation, analgesic compositions, anti-inflammatory compositions, etc. In non-limiting embodiments, the biocompatible liquid is one or more of polyethylene glycol (PEG), water, saline, dimethyl sulfoxide (DMSO), ethanol, isopropanol povidone-iodine, and chlorhexidine gluconate (CHG).

Those of skill in the art will appreciate that the various components described above may be included in various amounts (of each component) and various ratios (of each component to another or to the total of the liquid monomer mixture or final polymeric material). As described below, in non-limiting embodiments a total solids content of the liquid monomer mixture (acrylate(s), reactive component(s), acid(s), porogen(s), and/or photoinitiator(s) in solvent) may be in a range of about 10 wt % to about 70 wt %, optionally about 20 wt % to about 50 wt %, all values and subranges therebetween inclusive. In non-limiting embodiments, a monomer mixture useful in forming the polymeric materials described herein may include about 1 wt % to about 10 wt %, optionally about 3 wt % to about 7 wt %, optionally about 6 wt %, optionally about 5.6 wt %, optionally about 3 wt %, optionally about 3.4 wt % (all values and subranges therebetween inclusive) of one or more liquid crystal diacrylates; about 1 wt % to about 10 wt %, optionally about 2 wt % to about 5 wt %, optionally about 3 wt %, optionally about 2.8 wt %, optionally about 2 wt %, optionally about 1.7 wt % (all values and subranges therebetween inclusive) of one or more liquid crystal monoacrylates; about 2 wt % to about 10 wt %, optionally about 4 wt % to about 8.5 wt %, optionally about 8 wt %, optionally about 8.3 wt %, optionally about 5 wt % (all values and subranges therebetween inclusive) of one or more responsive components; about 15 wt % to about 45 wt %, optionally about 20 wt % to about 40 wt %, optionally about 35 wt %, optionally about 33 wt %, optionally about 33.3 wt %, optionally about 20 wt % (all values and subranges therebetween inclusive) of one or more acids or acid derivatives; about 1 wt % to about 5 wt %, optionally about 1 wt %, optionally about 2 wt % to about 3 wt %, optionally about 2 wt % (all values and subranges therebetween inclusive) of one or more photoinitiators; and/or about 30 wt % to about 80 wt %, optionally about 40 wt % to about 70 wt %, optionally about 50 wt %, optionally about 49 wt %, optionally about 70 wt %, optionally about 69 wt % (all values and subranges therebetween inclusive) of one or more porogens.

In non-limiting embodiments, the polymeric material described herein exhibits anisotropy. In terms of the function of the above-described polymeric materials, without wishing to be bound by the theory, the responsive materials described above, when included with the various other components described herein, provide for liquid crystal polymer networks (LCNs) as the polymeric material, which can exhibit anisotropic deformation due to their unique anisotropic properties in response to external stimulus, e.g., light, temperature, and electric field (see, e.g., White et al., Programmable and Adaptive Mechanics with Liquid Crystal Polymer Networks and Elastomers. Nat. Mater. 2015, 14, 1087-1098, the content of which is incorporated herein by reference in its entirety). The original study on linear thermal expansion of LCNs shows that the LCNs with a uniaxial planar alignment tend to expand perpendicular to the molecular director and contract parallel to the director. For light-induced deformation of the LCNs, typically, the principle is based on the so-called photochemical effect. The photochemical effect is based on the light-triggered trans-to-cis isomerization of an added azobenzene molecule. The azobenzene moiety may be provided by reactive groups similar to that of the liquid crystal monomers, so that it can be covalently embedded into the network after polymerization. Being co-aligned with the mesogenic units of the LCNs, the trans-cis isomerization of azobenzene disturbs the molecular order of the LCNs, which results in an anisotropic macroscopic deformation. Besides using the light trigger, the application of an electric field at a high frequency can also lead to a macroscopic anisotropic deformation of LCNs. In such non-limiting embodiments, a polar group with a large dipole moment that can react to the alternating electric field may be used.

Based on these principles, in the liquid-filled porous LCNs system, when applying an external stimulus, the LCNs can exhibit a macroscopic anisotropic deformation and therefore squeeze and release the biocompatible liquid. In the light-driven system, the principle is based on the photo-isomerization of the copolymerized azobenzene moiety. More specifically, a liquid-infused liquid crystal polymer coating with a homeotropic alignment is fabricated on a substrate. The coating is copolymerized with a light-responsive azobenzene molecule. Under a UV light illumination, the azobenzene moiety transforms from a rod-like trans-state to a bent cis-state, which creates a contraction force along the molecular alignment of the coating. This force is then exerted on the liquid crystal polymer network, which results in a reduction of the molecular order of the polymer network. Consequently, the disordered polymer network can squeeze, repel and release the stored liquid. When illuminating with visible light, the azobenzene can relax back to the stable trans-state, which creates a suction force. In combination with the elasticity of the polymer network, the released liquid at the surface can be reabsorbed.

In non-limiting embodiments, under the illumination of UV light in the range of absorption band of trans isomer of azobenzene, e.g. at a wavelength of 365 nm, the biocompatible liquid can be squeezed, repelled, and released to the surface of the coating. When illuminating with visible light in the range of absorption band of cis isomer of azobenzene, e.g. at a wavelength of 455 nm, the released liquid at the surface can be reabsorbed by the coating. Therefore, the coating can be refilled. The release and up-taking of the biocompatible liquid are completely reversible. And this reversible process can be repeated for multiple cycles without the significant loss of liquid due to evaporation.

When the liquid-infused coating/device is exposed to the air for quite a long time, e.g., a few hours, the stored liquid can escape from the polymer network due to the evaporation. In this case, the dry coating can still be refilled by absorption of some extra liquids which are sprayed onto the surface of the coating. The absorption can be based on three principles: (1) diffusion of liquid based on chemical potential; (2) capillary force induced by the pores within the polymer network; (3) possible extra capillary force induced by the isomerization of azobenzene moiety from cis-state to trans-state under the exposure to blue light.

In the case of the electro-induced liquid secretion and up-taking, the liquid secretion is based on liquid diffusion, electro-thermal effect, and deformation of the liquid crystal polymer network under the influence of alternating electric field at radio frequency (RF). When switching off the RF field, the liquid up-taking takes place, which is induced by capillary force in combination with the polymer elasticity.

Also provided herein are medical articles coated on at least one surface with a polymeric material as described herein. Any medical article may be coated with the polymeric materials, but, in particular implementations, the present disclosure contemplates medical articles that may benefit from the above-described ability of the materials to hold, release, and/or re-absorb biocompatible liquids. In non-limiting embodiments, the medical article is a medical article that is attached to a patient's skin. In non-limiting embodiment, the medical article is a medical tape, bandage, and/or wound covering. In non-limiting embodiments, the medical article, such as a tape, bandage, and/or wound covering, includes a substrate, optionally a flexible substrate, the polymeric material as described herein, and an adhesive. Suitable substrates for use in wound coverings/tapes are known to those of skill in the art and may include one or more of a polyester, a polyurethane, an olefin, a polyamide, an acrylic polymer, an ethylene-vinyl acetate, a silicone, a foam, a fluoropolymer, a paper, a non-woven polymeric material, a polyether, and derivatives thereof, may be transparent, translucent, or opaque, and may be formed into any suitable shape. In non-limiting embodiments, the polymeric material is arranged between the substrate and the adhesive. Suitable adhesives for medical applications are known to those of skill in the art, and may include one or more of 2-ethylhexyl acrylate, acrylic acid, isobutyl methacrylate, poly(ethylene glycol) methyl ether methacrylate, and poly(ethylene glycol) diacrylate, cyanoacrylate, silicone, polyisobutylene.

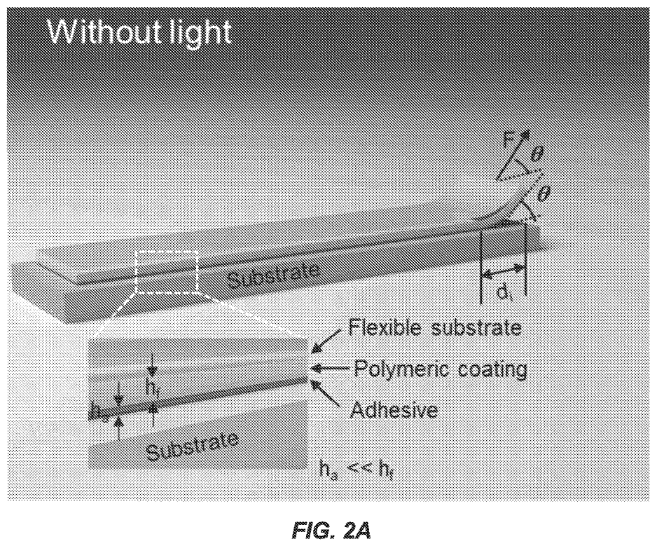

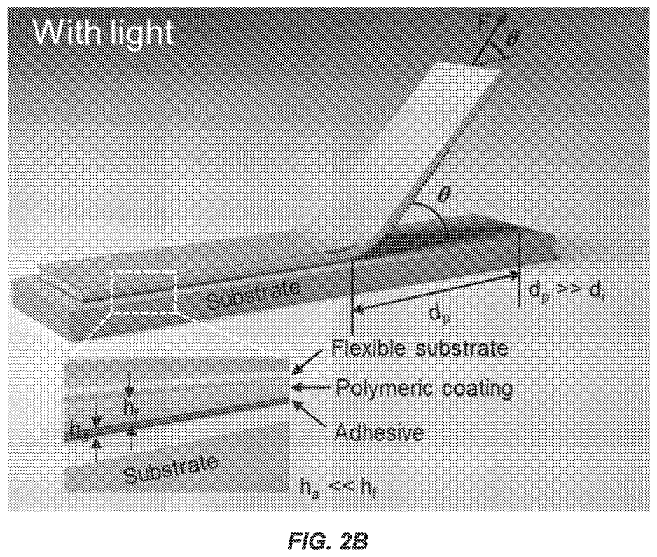

In non-limiting embodiments, the adhesive is porous, such that the biocompatible liquid received and/or held within the polymeric material can flow through the adhesive to contact, for example, a wearer's skin. In non-limiting embodiments, the surface area of any adhesive layer is smaller than the surface area of a polymeric material layer. Without wishing to be bound by the theory, the biocompatible liquid aids in the removal of the adhesive-containing substrate from the skin, reducing irritation and/or pain that would otherwise be caused, and potential advantages of using polymeric material so loaded include, without limitation, tunable surface adhesion (e.g., strong adhesion before liquid release and easily peeling off after liquid release without damaging the substrate, the wound, or the skin), possibilities to aid wound treatment (e.g., based on the releasing of a therapeutic composition carried by the liquid), and/or being reusable (e.g., based on the refilling of secreting liquids). In non-limiting embodiments, the biocompatible liquid includes an antiseptic, antibacterial, bactericidal, antifungal, and/or antiviral composition. In non-limiting embodiments, the biocompatible liquid includes one or more analgesic and/or anti-inflammatory compositions. The released liquid can then penetrate into the interface between the adhesive layer and the skin surface, reducing friction. Under a particular force, this polymeric plaster can be peeled off (as shown in FIG. 2B). In the case of depletion of the stored biocompatible liquid, the polymeric plaster can be refilled with the same liquid species or a different species. Thus, the polymeric plaster can be reused multiple times, enabling inspection and treatment for a variety of medical applications, such as dressings, securement devices, bandages, and other devices that are temporarily affixed for medical applications.

In non-limiting embodiments, the medical article is a device that is used in venous access, such as a cannula, a catheter, a hub, a connector (e.g., a luer connector, a needle free connector (NFC)), and/or any instrument that may be introduced into the vasculature or come into contact with blood. In non-limiting embodiments, an inner surface, lumen, and/or any portion of the device that comes into contact with blood may be coated with a polymeric material as described here. It is a solution to create adaptive responsive materials for lock/self-cleaning applications with the ability to control secretion and absorption of disinfectant/self-cleaning liquids such as but not limited to CHG, DMSO, IPA, Ethanol, etc. In non-limiting embodiments, the medical article, which may be formed of polyethylene terephthalate (PET), polycarbonate (PC), polyurethane, polytetrafluoroethylene (PTFE), polystyrene, silicone, and combinations thereof, may be surface grafter to improve adherence of the polymeric material described herein.

Also provided herein is a method of making a medical article coated on at least one surface thereof with a polymeric material as described herein. The method may include steps of dissolving one or more acrylates (e.g., liquid crystal acrylates) as described herein together with a responsive component as described herein, an acid as described herein, a porogen as described herein, and a photoinitiator as described herein in a solvent. Suitable solvents for preparing liquid monomer mixtures of acrylates are known to those of skill in the art, and may include tetrahydrofuran (THF), dichloromethane (DCM), 2-Methyltetrahydrofuran, diethylether, 1,2-dimethoxyethane (DME) and 1,4-dioxane. The liquid monomer mixture may then be applied to a substrate, using any suitable method. In non-limiting embodiments, a cell method or a spin coating method may be used. Thereafter, the solvent may be removed (e.g., the liquid monomer mixture may be dried), and the substrate with the monomer mixture thereon may be subject to polymerization conditions, for example by exposing the mixture to UV light. Thereafter, the polymerized material may be immersed or washed with a solvent to remove the porogen, leaving behind a porous polymeric material. Residual hydrogen bonds may be broken, and polymer salts may be formed, by immersing or washing the porous polymeric material with an alkaline solution. Suitable alkaline solutions are known to those of skill in the art, and may include potassium hydroxide (KOH), sodium hydroxide (NaOH), and ammonium hydroxide (NH₄OH). A biocompatible liquid (or combinations thereof) may then be loaded into the porous polymeric material by immersing the material in the biocompatible liquid.

Also provided herein is a method of using a medical article coated on at least one surface thereof with a polymeric material as described herein. As described above, the medical article may be any suitable medical article, including, without limitation, a cannula, a catheter, a hub, a connector (e.g., a luer connector, a needle free connector (NFC)), and/or any instrument that may be introduced into the vasculature or come into contact with blood. The method may include the steps of providing a medical article coated on at least one surface thereof with a polymeric material as described herein, and exposing the medical article to visible light and/or ultraviolet light to cause the polymeric material to exhibit a first orientation in which a biocompatible liquid as described herein is received within the pores, and a second orientation in which the biocompatible liquid is expelled from the pores. As described herein, in non-limiting embodiments the polymeric coating is configured to exhibit the first orientation when exposed to visible light and to exhibit the second orientation when exposed to ultraviolet light.

### Example 1

A cell is constructed by two glass slides: one is coated with 3-(trimethoxysilyl) propyl methacrylate to enhance surface adhesion, and the other is coated with a polyimide layer which provides a homeotropic alignment. The coating thickness is controlled by the defined spacers. The size of the spacer is in the range of 5 to 50 μm. The liquid crystal monomer mixture is filled in an empty cell by capillary force at the isotropic phase. After 20 min isothermal hold, the mixture is cooled down to its smectic phase, at which time the mixture is polymerized under UV illumination. A cut-off filter that allows light with a wavelength of >400 nm is used and placed in between the cell and the UV light source. After photopolymerization, the glass slide can be detached by using a razor blade. The coating is supported by the glass slide.

To remove the porogen, the coating is immersed into a solvent bath for 24 h. The solvent used includes, but is not limited to, cyclohexane and ethanol. The residual of the solvent is removed by evaporation at room temperature.

To break hydrogen bonds and form polymer salts, the coating is immersed in an alkaline bath. The alkaline solution used includes, but is not limited to, KOH solution. The concentration of KOH solution is in the range of 0.05 to 0.5 M. The immersing time is in the range of 30 s to 60 min

After base treatment, the coating is transferred to a liquid reservoir containing disinfectant/self-cleaning liquids including, but not limited to, DMSO, ethanol, isopropanol, or blends. The coating is refilled for 24 h.

### Example 2

A glass plate is coated with 3-(trimethoxysilyl) propyl methacrylate to enhance surface adhesion. The liquid crystal monomer mixture is dissolved in THF. The concentration of the solids in the solution ranges from 20 wt % to 50 wt %. The solution is coated onto the glass plate using a spin-coater. The spin-coating parameters are: (1) the speed is in the range of 300 rpm to 1000 rpm, depending on the concentration of the solid; (2) the acceleration is in the range of 100 rpm/s to 500 rpm/s. After the spin-coating process, the coating is transferred to a hotstage maintained at a temperature of 90° C. to evaporate THF for 5 min. The monomer coating is then slowly cooled down to its smectic phase. Depending on the composition of the monomer mixture, the temperature of the smectic phase varies from sample to sample. At the temperature of the smectic phase, the sample is photo-polymerized under UV illumination. A cut-off filter that allows light with a wavelength of >400 nm is used and placed in between the cell and the UV light source.

To remove the porogen, the coating is immersed into a solvent bath for 24 h. The solvent used includes, but is not limited to, cyclohexane and ethanol. The residual of the solvent is removed by evaporation at room temperature.

To break hydrogen bonds and form polymer salts, the coating is immersed in an alkaline bath. The alkaline solution used includes, but is not limited to, KOH solution. The concentration of KOH solution is in the range of 0.05 to 0.5 M. The immersing time is in the range of 30 sec to 60 min.

After base treatment, the coating is transferred to a liquid reservoir containing disinfectant/self-cleaning liquids including, but not limited to DMSO, ethanol, isopropanol, or blends. The coating is refilled for 24 h.

### Example 3

A cell is constructed by two slides: one is a flexible polymer film which is coated with 3-(trimethoxysilyl) propyl methacrylate to enhance surface adhesion; and the other is a glass slide which is coated with polyimide layer which provides a homeotropic alignment. In this case, the substrate used for supporting coating is a flexible polymer film, including, but not limited to, polyethylene terephthalate (PET). The PET may be coated with a thin layer of indium tin oxide to modify surface chemistry. The coating thickness is controlled by the defined spacers. The size of the spacer is in the range of 5 to 50 μm. The chemicals used for the formation of the polymer coating are the same as those used in 1. The liquid crystal monomer mixture is filled in an empty cell by capillary force at the isotropic phase. After 20 min isothermal, the mixture is cooled down to its smectic phase, at which time the mixture is polymerized under UV illumination. A cut-off filter that allows light with a wavelength of >400 nm is used and placed in between the cell and the UV light source. After photopolymerization, the glass slide can be detached by using a razor blade. The coating is supported by the flexible polymer film.

To remove the porogen, the coating is immersed into a solvent bath for 24 h. The solvent used includes but is not limited to cyclohexane and ethanol. The residual of the solvent is removed by evaporation at room temperature.

To break hydrogen bonds and form polymer salts, the coating is immersed in an alkaline bath. The alkaline solution used includes, but is not limited to, KOH solution. The concentration of KOH solution is in the range of 0.05 to 0.5 M. The immersing time is in the range of 30 sec to 60 min.

After base treatment, the coating is transferred to a liquid reservoir containing disinfectant/self-cleaning liquids including, but not limited to, DMSO, ethanol, isopropanol. The coating is refilled for 24 h.

The refilled coating is then ready to trigger liquid secretion and up-taking upon light illumination. To prevent liquid evaporation from the top surface of the coating, a sealing layer can be used. The sealing layer may be an adhesive layer, which can bring a desired adhesion force of the coating when it is in contact with the measuring substrate or the skin. To allow liquid release at the surface of the coating, the adhesive layer may contain a certain amount of pores; that is, the surface area of the adhesive layer is smaller than that of the polymeric coating.

### Example 4

A PET flexible film, coated with a thin layer of indium tin oxide is treated with a thin layer of 3-(trimethoxysilyl) propyl methacrylate to enhance surface adhesion using a coating technique including but not limited to spin-coating, dip-coating method, and chemical vapor deposition. The liquid crystal monomer mixture is dissolved in THF. The concentration of the solids in the solution ranges from 20 wt % to 50 wt %. The solution is coated onto the PET film using a spin-coater. The spin-coating parameters are: (1) the speed is in the range of 300 rpm to 1000 rpm, depending on the concentration of the solid; (2) the acceleration is in the range of 100 rpm/s to 500 rpm/s. After the spin-coating process, the coating is transferred to a hotstage at a temperature of 90° C. to evaporate THF for 5 min. The monomer coating is then slowly cooled down to its smectic phase. Depending on the composition of the monomer mixture, the temperature of the smectic phase varies from sample to sample. At the temperature of the smectic phase, the sample is photo-polymerized under UV illumination. A cut-off filter that allows light with a wavelength of >400 nm is used and placed in between the cell and the UV light source.

To remove the porogen, the coating is immersed into a solvent bath for 24 h. The solvent used includes but is not limited to cyclohexane and ethanol. The residual of the solvent is removed by evaporation at room temperature.

To break hydrogen bonds and form polymer salts, the coating is immersed in an alkaline bath. The alkaline solution used includes, but is not limited to, KOH solution. The concentration of KOH solution is in the range of 0.05 to 0.5 M. The immersing time is in the range of 30 s to 60 min.

After base treatment, the coating is transferred to a liquid reservoir containing disinfectant/self-cleaning liquids including, but not limited to, DMSO, ethanol, isopropanol. The coating is refilled for 24 h.

The refilled coating is then ready to trigger liquid secretion and up-taking upon light illumination. To prevent liquid evaporation from the top surface of the coating, a sealing layer can be used. The sealing layer may be an adhesive layer, which can bring a desired adhesion force of the coating when it is in contact with the measuring substrate or the skin. To allow liquid release at the surface of the coating, the adhesive layer should contain a certain amount of pores; that is, the surface area of the adhesive layer is smaller than that of the coating.

### Example 5

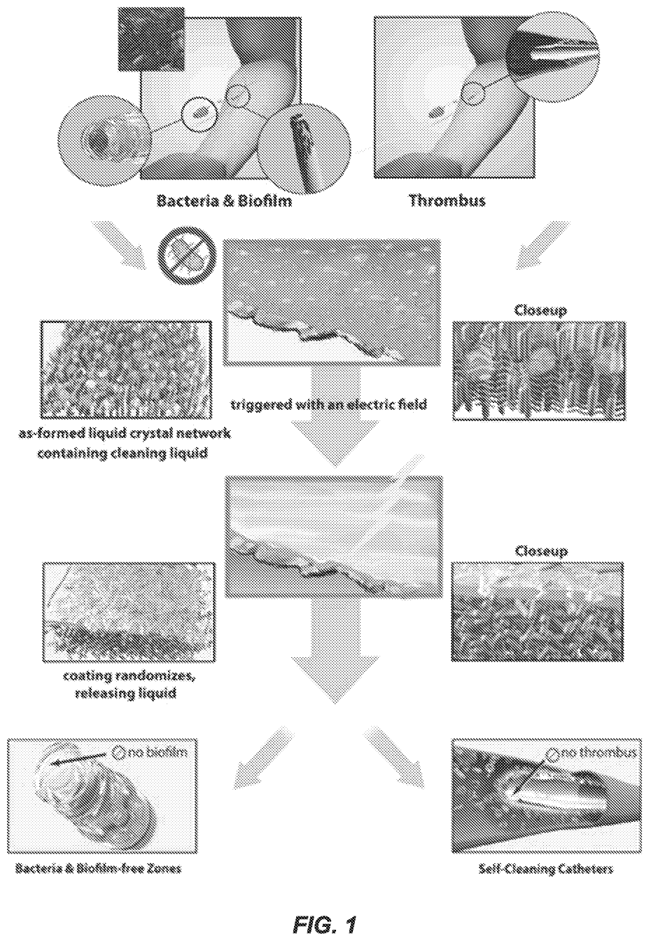

The polymeric coatings generated in Examples 3 and 4 may be utilized on a medical article. A schematic is shown in FIG. 1. A catheter needle-free connector (NFC) or lumen, or any other medical device, can be coated using liquid crystal polymer. Catheter connector or lumen coating component materials such as Polyethylene terephthalate (PET), Polycarbonate (PC), Polyurethane (PU), PTFE, Polystyrene, Silicone, and others can be surface grafted to enhance LCN coating adherence (list down two-three grafting kinds).

It should be noted that the schematic form of adaptive responsive materials for lock/self-cleaning applications with controlled secretion and uptake for medical device application is not limited to the illustrated example, and any medical device or non-medical device that is difficult to clean with standard procedures and requires disinfection or self-cleaning (FIG. 1, top panel) can be adopted based on purposes to any length and size. Under UV illumination (FIG. 1, middle panel), the coating releases liquid to the surface via the pores of the lumen or catheter surface. Under UV illumination, the coating enables liquid to be ejected to the surface through the pores of the LCN coating to the surface of lumen or catheter surface. In response to an external stimulus, such as light, electric field, or temperature, the LCN coating may release secretion and absorption liquids multiple times, forming droplets and establishing a liquid layer at the interfacial surface region (FIG. 01). As a result, the catheter/lumen surface will self-clean or disinfect (FIG. 1, bottom panel). Furthermore, the polymeric substrate may be coated on any medical device, including but not limited to dressings, securement devices, bandages, and so on, that may require multiple switches on and off, allowing inspection and treatment for a wide range of medical applications. This polymeric substrate or coating can also be applied to various medical devices for purposes such as temporary attachment, medication distribution, self-cleaning, and so on.

### Example 6

A cell is constructed by two glass slides: one is coated with 3-(trimethoxysilyl) propyl methacrylate to enhance surface adhesion, and the other is coated with a polyimide layer which provides a homeotropic alignment. The coating thickness is controlled by the defined spacers. The size of the spacer is in the range of 5 to 50 μm. The liquid crystal monomer mixture is filled in an empty cell by capillary force at the isotropic phase. After 20 min isothermal hold, the mixture is cooled down to its smectic phase, at which time the mixture is polymerized under UV illumination. A cut-off filter that allows light with a wavelength of >400 nm is used and placed in between the cell and the UV light source. After photopolymerization, the glass slide can be detached by using a razor blade. The coating is supported by the glass slide.

To remove the porogen, the coating is immersed into a solvent bath for 24 h. The solvent used includes, but is not limited to, cyclohexane and ethanol. The residual of the solvent is removed by evaporation at room temperature.

To break hydrogen bonds and form polymer salts, the coating is immersed in an alkaline bath. The alkaline solution used includes, but is not limited to, KOH solution. The concentration of KOH solution is in the range of 0.05 to 0.5 M. The immersing time is in the range of 30 sec to 60 min.

After base treatment, the coating is transferred to a liquid reservoir containing biocompatible liquid including, but not limited to, polyethylene glycol, glycerol, DMSO, water, ethanol, isopropanol, or blends. The use of blend is to reduce the viscosity of the polymer ethylene glycol but also to lower the evaporation of the liquids. The coating is refilled for 24 h.

### Example 7

A glass plate is coated with 3-(trimethoxysilyl) propyl methacrylate to enhance surface adhesion. The liquid crystal monomer mixture is dissolved in THF. The concentration of the solids in the solution ranges from 20 wt % to 50 wt %. The solution is coated onto the glass plate using a spin-coater. The spin-coating parameters are: (1) the speed is in the range of 300 rpm to 1000 rpm, depending on the concentration of the solid; (2) the acceleration is in the range of 100 rpm/s to 500 rpm/s. After the spin-coating process, the coating is transferred to a heat source, for example a hotstage/hotplate, and maintained at a temperature of 90° C. to evaporate THF for 5 min. The monomer coating is then slowly cooled down to its smectic phase. Depending on the composition of the monomer mixture, the temperature of the smectic phase varies from sample to sample. At the temperature of the smectic phase, the sample is photo-polymerized under UV illumination. A cut-off filter that allows light with a wavelength of >400 nm is used and placed in between the cell and the UV light source.

To remove the porogen, the coating is immersed into a solvent bath for 24 h. The solvent used includes, but is not limited to, cyclohexane and ethanol. The residual of the solvent is removed by evaporation at room temperature.

To break hydrogen bonds and form polymer salts, the coating is immersed in an alkaline bath. The alkaline solution used includes, but is not limited to, KOH solution. The concentration of KOH solution is in the range of 0.05 to 0.5 M. The immersing time is in the range of 30 sec to 60 min.

After base treatment, the coating is transferred to a liquid reservoir containing biocompatible liquid including, but not limited to, polyethylene glycol, DMSO, water, ethanol, isopropanol, or blends. The use of blend is to reduce the viscosity of the polymer ethylene glycol but also to lower the evaporation of the liquids. The coating is refilled for 24 h.

### Example 8

A cell is constructed by two slides: one is a flexible polymer film which is coated with 3-(trimethoxysilyl) propyl methacrylate to enhance surface adhesion; and the other is a glass slide which is coated with polyimide layer which provides a homeotropic alignment. In this case, the substrate used for supporting coating is a flexible polymer film, including, but not limited to, polyethylene terephthalate (PET). The PET may be coated with a thin layer of indium tin oxide to modify surface chemistry. The coating thickness is controlled by the defined spacers. The size of the spacer is in the range of 5 to 50 μm. The chemicals used for the formation of the polymer coating are the same as those used in 1. The liquid crystal monomer mixture is filled in an empty cell by capillary force at the isotropic phase. After 20 min isothermal, the mixture is cooled down to its smectic phase, at which temperature the mixture is polymerized under UV illumination. A cut-off filter that allows light with a wavelength of >400 nm is used and placed in between the cell and the UV light source. After photopolymerization, the glass slide can be detached by using a razor blade. The coating is supported by the flexible polymer film.

To remove the porogen, the coating is immersed into a solvent bath for 24 h. The solvent used includes but is not limited to cyclohexane and ethanol. The residual of the solvent is removed by evaporation at room temperature.

To break hydrogen bonds and form polymer salts, the coating is immersed in an alkaline bath. The alkaline solution used includes, but is not limited to, KOH solution. The concentration of KOH solution is in the range of 0.05 to 0.5 M. The immersing time is in the range of 30 sec to 60 min.

After base treatment, the coating is transferred to a liquid reservoir containing biocompatible liquid including, but not limited to, polyethylene glycol, DMSO, water, ethanol, isopropanol. The use of blend is to reduce the viscosity of the polymer ethylene glycol but also to lower the evaporation of the liquids. The coating is refilled for 24 h.

The refilled coating is then ready to trigger liquid secretion and up-taking upon light illumination. To prevent liquid evaporation from the top surface of the coating, a sealing layer can be used. The sealing layer may be an adhesive layer, which can bring a desired adhesion force of the coating when it is in contact with the measuring substrate or the skin. To allow liquid release at the surface of the coating, the adhesive layer should contain a certain amount of pores; that is, the surface area of the adhesive layer is smaller than that of the coating.

### Example 9

A PET flexible film, coated with a thin layer of indium tin oxide is treated with a thin layer of 3-(trimethoxysilyl) propyl methacrylate to enhance surface adhesion using a coating technique including but not limited to spin-coating, dip-coating method, and chemical vapor deposition. The liquid crystal monomer mixture is dissolved in THF. The concentration of the solids in the solution ranges from 20 wt % to 50 wt %. The solution is coated onto the PET film using a spin-coater. The spin-coating parameters are: (1) the speed is in the range of 300 rpm to 1000 rpm, depending on the concentration of the solid; (2) the acceleration is in the range of 100 rpm/s to 500 rpm/s. After the spin-coating process, the coating is transferred to a hotstage at a temperature of 90° C. to evaporate THF for 5 min. The monomer coating is then slowly cooled down to its smectic phase. Depending on the composition of the monomer mixture, the temperature of the smectic phase varies from sample to sample. At the temperature of the smectic phase, the sample is photo-polymerized under UV illumination. A cut-off filter that allows light with a wavelength of >400 nm is used and placed in between the cell and the UV light source.

To remove the porogen, the coating is immersed into a solvent bath for 24 h. The solvent used includes but is not limited to cyclohexane and ethanol. The residual solvent is removed by evaporation at room temperature.

To break hydrogen bonds and form polymer salts, the coating is immersed in an alkaline bath. The alkaline solution used includes, but is not limited to, KOH solution. The concentration of KOH solution is in the range of 0.05 to 0.5 M. The immersing time is in the range of 30 sec to 60 min.

After base treatment, the coating is transferred to a liquid reservoir containing biocompatible liquid including, but not limited to, polyethylene glycol, DMSO, water, ethanol, isopropanol. The use of blend is to reduce the viscosity of the polymer ethylene glycol but also to lower the evaporation of the liquids. The coating is refilled for 24 h.

The refilled coating is then ready to trigger liquid secretion and up-taking upon light illumination. To prevent liquid evaporation from the top surface of the coating, a sealing layer can be used. The sealing layer may be an adhesive layer, which can bring a desired adhesion force of the coating when it is in contact with the measuring substrate or the skin. To allow liquid release at the surface of the coating, the adhesive layer should contain a certain amount of pores; that is, the surface area of the adhesive layer is smaller than that of the coating.

### Example 10

The coatings prepared in Example 8 and Example 9 may be combined in a medical device with a substrate and an adhesive. The materials used for the fabrication of adhesive include, but are not limited to, 2-ethylhexyl acrylate, acrylic acid, isobutyl methacrylate, poly (ethylene glycol) methyl ether methacrylate, or poly(ethylene glycol) diacrylate. The polymer adhesives are synthesized by photopolymerization. More in detail, a low percentage of photo-initiator is added to the monomer mixture mentioned above, and the mixture is polymerized under UV illumination for 30 min with a nitrogen gas atmosphere. After photopolymerization, the polymer is immersed into a solvent to remove the residual of unreacted monomers and photo-initiator. The final polymer adhesive is safe to use on human or animal skin.

The design of the adhesion tunable polymer material is shown schematically in FIGS. 2A and 2B. The liquid crystal polymer coating is supported by a flexible PET film. On the top of the coating, a pressure-sensitive adhesive layer is coated. The top adhesive can be coated by spin-coating or spray-coating a mixture of synthesized adhesive with a solvent. This adhesive layer also acts as a protective layer and a sealing layer. The adhesive layer contains pores with a size in the range of 5 μm to 2 mm. The thickness of the adhesive layer is in the range of 500 nm to 5 μm. It should be noted that the planar-view form of the adhesion-tunable liquid-secreting polymeric material for medical device application is not limited to the illustrated example, and any acceptable shape (such as a circle, a square, a rectangle, or a trapezoid) can be adopted based on purposes to any length and size.

When in the absence of UV light illumination (FIG. 2A), the film cannot be peeled off the measuring substrate under a certain force with a certain tilted angle. Under UV illumination (FIG. 2B), the coating releases liquid to the surface via the pores of the adhesive layer. This results in a decrease in surface adhesion. Therefore, under a certain force with a certain tilted angle, the coating can be easily peeled off.

### Example 11

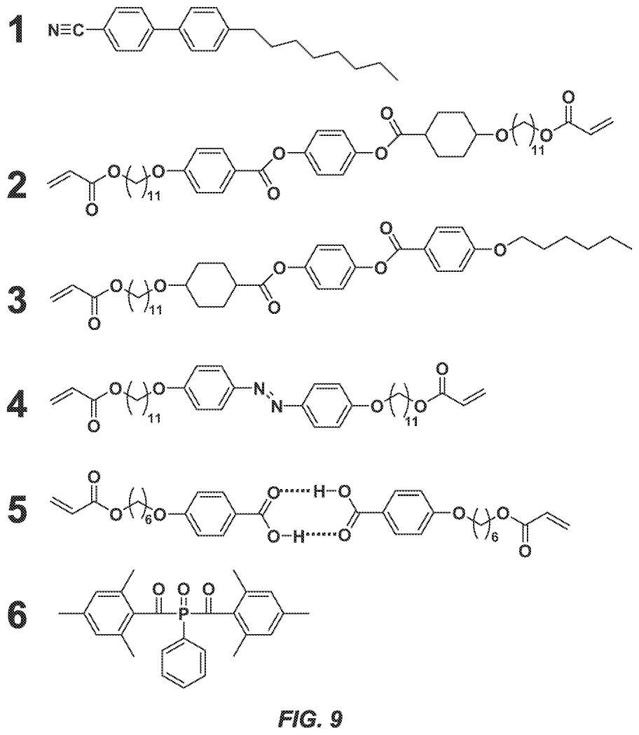

A liquid crystal monomer mixture is composed of 69 wt. % of molecule 1, 3.4 wt. % of molecule 2, 1.7 wt. % of molecule 3, 5.0 wt. % of molecule 4, 20 wt. % of molecule 5, and 1 wt. % of molecule 6. Chemical structures of molecule 1 to 6 are shown in FIG. 9. The monomers are mixed in THF to form a homogenous mixture. After evaporation of THE, the monomer mixture is filled in an empty cell at 80° C. at which the sample is held for 30 min. The cell is constructed by a PET substrate and a glass plate. The PET substrate, which is pre-coated with a thin ITO layer, is then coated with a layer of 3-(trimethoxysilyl) propyl methacrylate to improve adhesion force; and the glass plate is coated with a polyimide layer which provides homeotropic alignment (SE-5661, Nissan Chemical). Then the sample is cooled down to 30° C. and exposed to UV light to perform photopolymerization for 1 h. The polymerized coating is immersed into a cyclohexane bath for 24 h to remove porogen. Then the coating without porogen is transferred to a KOH solution with a concentration of 0.05 M and immersed for 1 min. Next, the coating is refilled with DMSO or a blend of DMSO with PEG in a volume ratio in the range of 10/90 to 100/0.

### Example 12

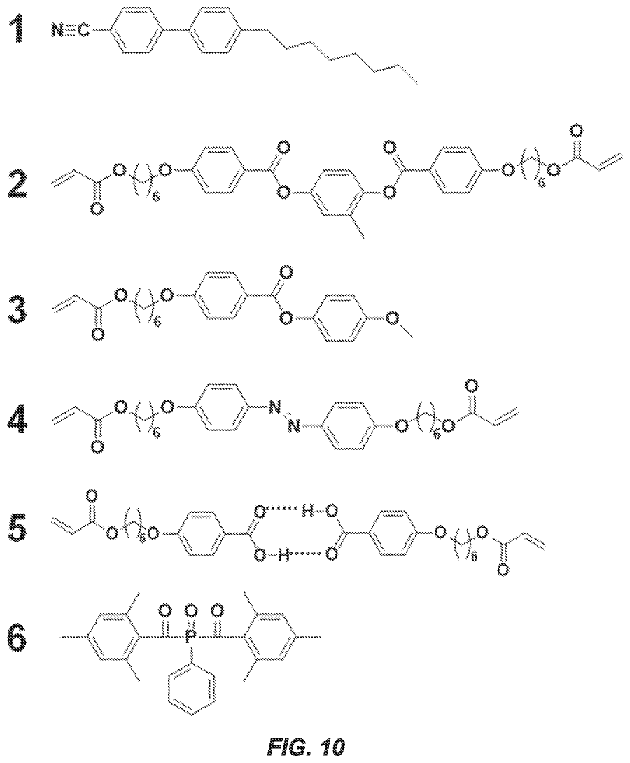

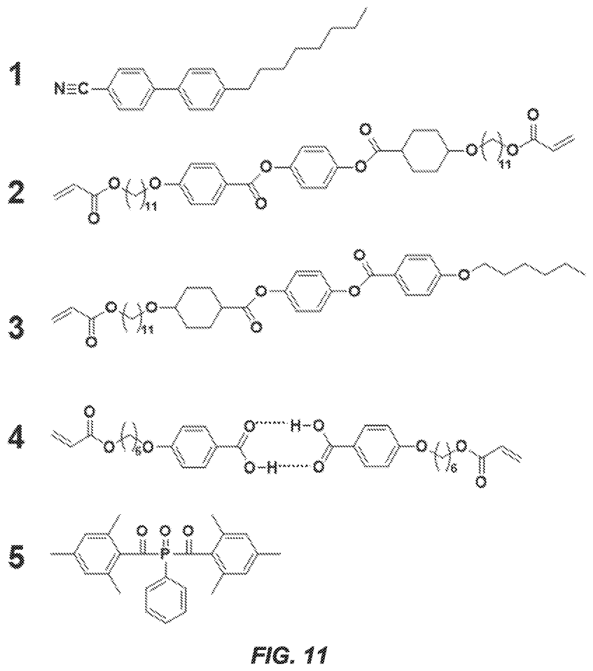

A different liquid crystal monomer mixture is composed of 49 wt % of molecule 1, 5.6 wt % of molecule 2, 2.8 wt % of molecule 3, 8.3 wt % of molecule 4, 33.3 wt % of molecule 5, and 2 wt % of molecule 6. The chemical structures of molecule 1 to 6 are shown in FIG. 10. The monomers are mixed in THF to form a homogenous mixture at a concentration of 50 wt %. After evaporation of THE, the monomer mixture is spin-coated onto an ITO-PET film. The ITO-PET plate is coated with an adhesion improving layer of 3-(trimethoxysilyl) propyl methacrylate. The spin-coating is performed at a speed of 500 rpm with an acceleration of 200 rpm/s. After spin-coating, the monomer coating is heated to 90° C. at which the sample is held for 10 min to evaporate the solvent. Then the sample is cooled down to 52° C. and exposed to UV light to perform photopolymerization for 1 h. The polymerized coating is immersed into a cyclohexane bath for 24 h to remove porogen. Then the coating without porogen is transferred to a KOH solution with a concentration of 0.1 M and immersed for 5 min. Next, the coating is refilled with a blend of PEG with water in a volume ratio ranging from 10/90 to 100/0.

When applying the polymeric material to the skin surface of a medical mannequin, prior to the release of liquid, the coating firmly adheres to the surface even under an exerted certain force. When the coating is exposed to a UV light at a dose of 0.4 J/cm², [1] the coating can be easily removed from the medical mannequin under an external force at 30° parallel to the surface of the medical mannequin. When applying the polymeric material to a glass substrate, prior to the release of liquid, the coating firmly adheres to the glass even under an exerted certain force. When the coating is exposed to a UV light at a dose of 0.5 J/cm², the coating can be easily removed from the substrate under an external force at 180° parallel to the surface of the substrate.

### Example 13

An alternative trigger for inducing liquid secretion and up-taking is the electric field. Differed from the light-driven system, the electro-driven liquid secretion is based on liquid diffusion, electro-thermal effect, and deformation of the liquid crystal polymer network under the influence of an alternating electric field at radio frequency (RF). When switching off the RF field, the liquid up-taking takes place, which is induced by capillary force in combination with the polymer elasticity. In this case, the chemistry of the liquid crystal polymer coating has to be modified. An example of a liquid crystal composition is given in FIG. 11. Molecule 1 is used as porogen but also to form the smectic structure. Liquid crystal diacrylate 1 and monoacrylate 2 are used to form the liquid crystal polymer network. Molecule 4 is a benzoic acid derivative, used for increasing the polymer polarity. Photo-initiator 5 is used for creating radicals and initiating polymerization.

### Example 14

A liquid crystal polymer coating is been fabricated by one of two alternate methods: a cell method or a spin-coating method. Each method is explained as follows:

Cell method. A cell is constructed by two slides: one flexible interdigitated electrode (IDE) substrate coated with Indium Tin Oxide (ITO) is coated with 3-(trimethoxysilyl) propyl methacrylate to enhance surface adhesion; and the other is a glass slide which is coated with polyimide layer which provides a homeotropic alignment. In this case, the substrate used for supporting coating is the flexible IDE polymer film, including, but not limited to, polyethylene terephthalate (PET). The coating thickness is controlled by the defined spacers. The size of the spacer is in the range of 5 to 50 μm. The liquid crystal monomer mixture is filled in an empty cell by capillary force at the isotropic phase. After 20 min isothermal, the mixture is cooled down to its smectic phase, at which temperature the mixture is polymerized under UV illumination. After photopolymerization, the glass slide can be detached by using a razor blade. The coating is supported by the flexible IDE substrate.

Spin-coating method. A PET flexible IDE substrate is treated with a thin layer of 3-(trimethoxysilyl) propyl methacrylate to enhance surface adhesion using a coating technique including but not limited to spin-coating, dip-coating method, and chemical vapor deposition. The liquid crystal monomer mixture is dissolved in THF. The concentration of the solids in the solution ranges from 20 wt. % to 50 wt. %. The solution is coated onto the PET substrate using a spin-coater. The spin-coating parameters are: (1) the speed is in the range of 300 rpm to 1000 rpm, depending on the concentration of the solid; (2) the acceleration is in the range of 100 rpm/s to 500 rpm/s. After the spin-coating process, the coating is transferred to a hot-stage at a temperature of 90° C. to evaporate THF for 5 min. The monomer coating is then slowly cooled down to its smectic phase. Depending on the composition of the monomer mixture, the temperature of the smectic phase varies from sample to sample. At the temperature of the smectic phase, the sample is photo-polymerized under UV illumination using a UV LED lamp.

To remove the porogen, the coating is immersed into a solvent bath for 24 h. The solvent used includes but is not limited to cyclohexane and ethanol. The residual solvent is removed by evaporation at room temperature. To break hydrogen bonds and form polymer salts, the coating is immersed in an alkaline bath. The alkaline solution used includes, but is not limited to, KOH solution. The concentration of KOH solution is in the range of 0.05 to 0.5 M. The immersing time is in the range of 30 s to 60 min. After base treatment, the coating is transferred to a liquid reservoir containing self-cleaning or disinfection liquids including, but not limited to, DMSO, ethanol, isopropanol. The use of blend is to reduce the viscosity of the polymer ethylene glycol but also to lower the evaporation of the liquids. For the initial coating, the coating is filled for 24 h. It is expected that optimization will reduce this time.

### Example 15

A liquid crystal polymer coating is fabricated by one of two alternate methods: a cell method or a spin-coating method. Each method is explained as follows:

Cell method. A cell is constructed by two glass slides: one with interdigitated electrodes (IDE) is coated with 3-(trimethoxysilyl) propyl methacrylate to enhance surface adhesion, and the other common glass is coated with a polyimide layer which provides a homeotropic alignment. The electrodes include but are not limited to indium tin oxide (ITO). The coating thickness is controlled by the defined spacers. The size of the spacer is in the range of 5 to 50 μm. The liquid crystal monomer mixture is filled in an empty cell by capillary force at the isotropic phase. After 20 min isothermal hold, the mixture is cooled down to its smectic phase, at which temperature the mixture is polymerized under UV illumination. After 1 h photopolymerization, the glass slide can be detached by using a razor blade. Finally, the coating is attached to the IDE glass substrate.

Spin-coating method. An IDE glass plate is coated with 3-(trimethoxysilyl) propyl methacrylate to enhance surface adhesion. The electrodes include but are not limited to ITO. The liquid crystal monomer mixture is dissolved in THF. The concentration of the solids in the solution ranges from 20 wt % to 50 wt %. The solution is coated onto the ITO glass plate using a spin-coater. The spin-coating parameters are: (1) the speed is in the range of 300 rpm to 1000 rpm, depending on the concentration of the solid; (2) the acceleration is in the range of 100 rpm/s to 500 rpm/s. After the spin-coating process, the coating is transferred to a hotstage maintained at a temperature of 90° C. to evaporate THF for 5 min. The monomer coating is then slowly cooled down to its smectic phase. Depending on the composition of the monomer mixture, the temperature of the smectic phase varies from sample to sample. At the temperature of the smectic phase, the sample is photo-polymerized under UV illumination using a UV LED lamp.

After photopolymerization for 1 h, the coating is immersed into a solvent bath for 24 h to remove the porogen. The solvent used includes, but is not limited to, cyclohexane and ethanol. The residual solvent is removed by evaporation at room temperature. To break hydrogen bonds and form polymer salts, the coating is immersed in an alkaline bath. The alkaline solution used includes, but is not limited to, KOH solution. The concentration of KOH solution is in the range of 0.05 to 0.5 M. The immersing time is in the range of 30 sec to 60 min, depending on the concentration of the benzoic acid derivative. After the base treatment, the coating is transferred to a liquid reservoir containing biocompatible liquid including, but not limited to, polymer ethylene glycol, DMSO, water, ethanol, isopropanol, or blends. The use of a blend is to reduce the viscosity of the polymer ethylene glycol but also to lower the evaporation of the liquids. The coating is refilled for 24 h.

### Example 16

A liquid crystal polymer coating is fabricated by one of two alternate methods: a cell method or a spin-coating method. Each method is explained as follows:

Cell method. A cell is constructed by two slides: one flexible IDE substrate coated with ITO is coated with 3-(trimethoxysilyl) propyl methacrylate to enhance surface adhesion; and the other is a glass slide which is coated with polyimide layer which provides a homeotropic alignment. In this case, the substrate used for supporting the coating is the flexible IDE polymer film, including, but not limited to, PET. The coating thickness is controlled by the defined spacers. The size of the spacer is in the range of 5 to 50 μm. The liquid crystal monomer mixture is filled into an empty cell by capillary force at the isotropic phase. After 20 min of isothermal, the mixture is cooled down to its smectic phase, at which temperature the mixture is polymerized under UV illumination. After photopolymerization, the glass slide can be detached by using a razor blade. The coating is supported by the flexible IDE substrate.

Spin-coating method. A PET flexible IDE substrate is treated with a thin layer of 3-(trimethoxysilyl) propyl methacrylate to enhance surface adhesion using a coating technique including but not limited to spin-coating, dip-coating method, and chemical vapor deposition. The liquid crystal monomer mixture is dissolved in THF. The concentration of the solids in the solution ranges from 20 wt % to 50 wt %. The solution is coated onto the PET substrate using a spin-coater. The spin-coating parameters are: (1) the speed is in the range of 300 rpm to 1000 rpm, depending on the concentration of the solid; (2) the acceleration is in the range of 100 rpm/s to 500 rpm/s. After the spin-coating process, the coating is transferred to a hotstage/hotplate at a temperature of 90° C. to evaporate THF for 5 min. The monomer coating is then slowly cooled down to its smectic phase. Depending on the composition of the monomer mixture, the temperature of the smectic phase varies from sample to sample. At the temperature of the smectic phase, the sample is photo-polymerized under UV illumination using a UV LED lamp.

To remove the porogen, the coating is immersed into a solvent bath for 24 h. The solvent used includes but is not limited to cyclohexane and ethanol. The residual solvent is removed by evaporation at room temperature. To break hydrogen bonds and form polymer salts, the coating is immersed in an alkaline bath. The alkaline solution used includes, but is not limited to, KOH solution. The concentration of KOH solution is in the range of 0.05 to 0.5 M. The immersing time is in the range of 30 sec to 60 min. After base treatment, the coating is transferred to a liquid reservoir containing biocompatible liquid including, but not limited to, polyethylene glycol, DMSO, water, ethanol, isopropanol. The use of a blend is to reduce the viscosity of the polymer ethylene glycol but also to lower the evaporation of the liquids. The coating is refilled for 24 h. A variety of solvents may be utilized, including DMSO, ethanol, isopropanol, including blends with larger molecules. Demonstration samples were made with polyethylene glycol blended with other smaller molecules, opening up the opportunity for select drugs and other active agents to be included for secretion and uptake.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

1. A medical article comprising:
a substrate;
a polymeric coating arranged on the substrate, the coating comprising at least one liquid crystal acrylate and a responsive component; and
a biocompatible liquid received within the polymeric coating.

2. The medical article of claim 1, wherein the polymeric coating is anisotropic.

3. The medical article of claim 1, wherein the liquid crystal acrylate comprises a liquid crystal diacrylate and a liquid crystal monoacrylate.

4. The medical article of claim 3, wherein the liquid crystal diacrylate has the following structure:

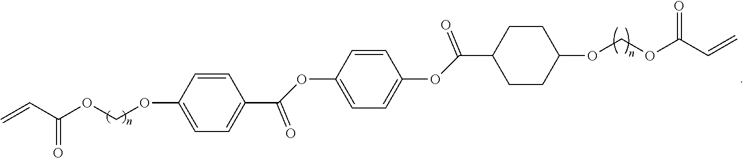

5. The medical article of claim 3, wherein the liquid crystal diacrylate has the following structure:

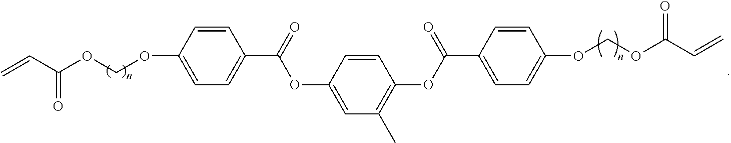

6. The medical article of claim 3, wherein the liquid crystal monoacrylate has the following structure:

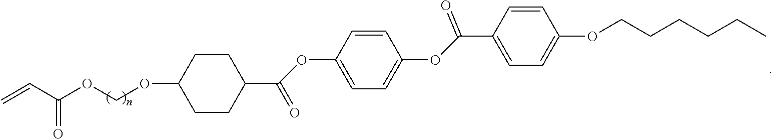

7. The medical article of claim 3, wherein the liquid crystal monoacrylate has the following structure:

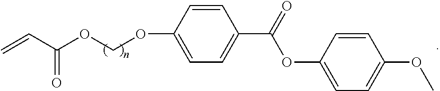

8. The medical article of claim 3, wherein the responsive component is a light-responsive component comprising a light-responsive diacrylate.

9. The medical article of claim 8, wherein the light-responsive component comprises an azobenzene moiety.

10. The medical article of claim 8, wherein the light-responsive diacrylate has the following structure:

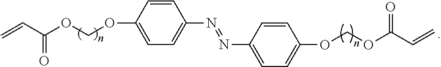

11. The medical article of claim 1, wherein the polymeric coating further comprises a carboxylic acid.

12. The medical article of claim 11, wherein the carboxylic acid is benzoic acid.

13. The medical article of claim 1, wherein the polymeric coating further comprises a photoinitiator.

14. The medical article of claim 13, wherein the photoinitiator contains phosphorous.

15. The medical article of claim 13, wherein the photoinitiator has the following structure:

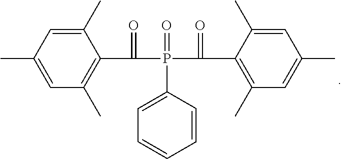

16. The medical article of claim 1, wherein the polymeric coating comprises a plurality of pores configured to receive a liquid therein.

17. The medical article of claim 16, wherein the biocompatible liquid is received within the pores of the polymeric coating.

18. The medical article of claim 17, wherein the biocompatible liquid comprises one or more of polyethylene glycol, water, saline, dimethyl sulfoxide (DMSO), ethanol, isopropanol, and chlorhexidine gluconate.

19. The medical article of claim 17, wherein the polymeric coating is configured to exhibit a first orientation in which the biocompatible liquid is received within the pores, and a second orientation in which the biocompatible liquid is expelled from the pores.

20. The medical article of claim 19, wherein the polymeric coating is configured to exhibit the first orientation when exposed to visible light and to exhibit the second orientation when exposed to ultraviolet light.

21. The medical article of claim 1, wherein the substrate comprises a catheter, a catheter hub, a luer connector, and/or a needle-free connector (NFC), and wherein the polymeric coating is arranged on an inner surface of the medical article.

22. The medical article of claim 1, wherein the substrate is optionally flexible, and the medical article comprises an adhesive layer arranged on the polymeric coating, such that the polymeric coating is arranged between the adhesive and the flexible substrate.

23. The medical article of claim 22, wherein the adhesive layer comprises a plurality of pores.

24. The medical article of claim 22, wherein the adhesive layer comprises one or more of 2-ethylhexyl acrylate, acrylic acid, isobutyl methacrylate, poly(ethylene glycol) methyl ether methacrylate, and poly(ethylene glycol) diacrylate.

25. The medical article of claim 22, wherein the substrate comprises one or more of a polyester, a polyurethane, an olefin, a polyamide, and acrylic polymer, an ethylene-vinyl acetate, a polyether, and derivatives thereof.

26.-49. (canceled)
